(19) United States
(12) Patent Application Publication
(10) Pub. No.: US 2013/0142849 A1
(43) Pub. Date: Jun. 6, 2013

(54) CONTROLLED RELEASE FORMULATIONS OF DRONEDARONE

(71) Applicants: Ashish Ashokrao Deshmukh, Pune (IN); Pravin Meghrajji Bhutada, Pune (IN); Sajeev Chandran, Pune (IN); Shirish Kulkarni, Pune (IN)

(72) Inventors: Ashish Ashokrao Deshmukh, Pune (IN); Pravin Meghrajji Bhutada, Pune (IN); Sajeev Chandran, Pune (IN); Shirish Kulkarni, Pune (IN)

(73) Assignee: LUPIN LIMITED, Mumbai, State of Maharashtra (IN)

(21) Appl. No.: 13/817,169

(22) PCT Filed: Aug. 13, 2011
(86) PCT No.: PCT/IB2011/001882

§ 371 Date: Feb. 15, 2013

(30) Foreign Application Priority Data
Aug. 17, 2010 (IN) 924/KOL/2010

(51) Int. Cl. A61K 47/32; A61K 47/38; A61K 31/35; A61K 47/34

(52) U.S. Cl. CPC A61K 47/38
USPC 424/400; 514/469

(57) ABSTRACT

The present invention relates to controlled release formulation of dronedarone or pharmaceutically acceptable salts, esters, metabolites, prodrugs or enantiomers thereof and controlled release polymers. The use of controlled release formulations of Dronedarone would improve the bioavailability and the patient compliance with reduction in number of dosages to be taken per day.

### FIELD OF THE INVENTION

The present invention relates to controlled release formulation for the delivery of an anti-arrhythmic drug. The controlled release formulation comprises of dronedarone or pharmaceutically acceptable salts, esters, metabolites, prodrugs or enantiomers thereof.

### BACKGROUND OF THE INVENTION

Cardiac arrhythmia is a term for any large and heterogeneous group of conditions in which there is abnormal electric activity in the heart. An arrhythmia is a disorder of the heart rateheart rate (pulse) or heart rhythm, such as beating too fast (tachycardia), too slow (bradycardia), or irregularly. Arrhythmias can be life-threatening medical emergencies which can result in cardiac arrest and sudden death.

Normally, the four chambers of the heart contract in very specific and coordinated manner. The electrical impulse that signals the heart to contract in a synchronized manner begins in the sinoatrial node (SA node) which is heart's natural pacemaker. The signal leaves the SA node and travels through the two upper chambers (atria). Then the signal passes through the atrioventricular node (AV node). Finally it passes through the lower chambers (ventricles). This path enables the chambers to contract in a coordinated fashion. Problems can occur anywhere along this conduction system, causing various arrhythmias. The examples include: Bradycardia—a slow heart rate due to problems with the SA node's pacemaker ability, or an interruption in energy movement (conduction) through the natural electrical pathways of the heart. Supraventricular tachycardia—a fast heart rate that originates in the upper chambers (atria). The most common are atrial fibrillation or flutter (a rapid heart rate that is not regular).and atrioventricular nodal reentry tachycardia (AVNRT). Ventricular tachycardia—a fast heart rate that originates in the lower chambers (ventricles).

The method of cardiac rhythm management depends firstly on whether or not the affected person is stable or unstable. Treatments may include physical maneuvers, medications, electricity conversion, or electro or cryo cautery. When an arrhythmia is serious, urgent treatment may be required to restore a normal rhythm. This may include: electrical “shock” therapy (defibrillation or cardioversion), implanting a temporary pacemaker to interrupt the arrhythmia medications given through a vein (intravenous). Medications are generally used to prevent and/or manage arrhythmia. There are many classes of antiarrhythmic medications, with different mechanisms of action which include Sodium Channel Blockers (Class I) e.g. Class IA—Quinidine (Quinidex), Procainamide (Pronestyl), Disopyramide (Norpace); Class IB—Lidocaine (Xylocalne), Tocamide (Tonocard), Mexiletine (Mexitil); Class IC— Encamide (Enkaid), Flecamide (Tambocor); Beta-Adrenergic Blockers (Class II)—Propranolol (Inderal), Acebutolol (Sectral), Esmolol (Brevibloc), Sotalol (Betapace); Drugs that Prolong Repolarization (Class III)—Dronedarone (Multaq), Amiodarone (Cordarone); Calcium Channel Blockers (Class IV)—Verapamil (Calan, Isoptin), Diltiazem (Cardizem), Mebefradil (Posicor); Miscellaneous—Adenosine (Adenocard), Digoxin (Lanoxin).

Dronedarone hydrochloride is N-{2-butyl-3-[4-(3-dibutylaminopropoxy)benzoyl]benzofuran-5-yl}methane sulfonamide, hydrochloride.

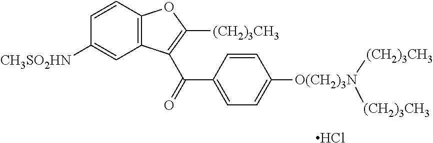

The effects of Dronedarone most likely result from its electrophysiological properties belonging to all four Vaughan-Williams classes. Dronedarone is a multichannel blocker inhibiting the potassium currents (including IK (Ach), IKur, IKr, IKs) and thus prolonging cardiac action potential and refractory periods (Class III). It also inhibits the sodium currents (Class Ib) and the calcium currents (Class IV). It non-competitively antagonizes adrenergic activities (Class II). Dronedarone works by altering currents passing through potassium, sodium, and calcium channels, thereby prolonging conduction in the heart. This helps maintain a regular heart rhythm or sinus rhythm and slows the heart rate.

The available dosage for Dronedarone is 400 mg oral tablet to be administered twice a day with meals. Dronedarone hydrochloride (Multaq®; Sanofi-Aventis) was approved to reduce the risk of cardiovascular hospitalization in patients with paroxysmal or persistent atrial fibrillation (AF) or atrial flutter (AFL), with a recent episode of AF/AFL and associated cardiovascular risk factors (i.e., age >70, hypertension, diabetes, prior cerebrovascular accident, left atrial diameter ≧50 mm or left ventricular ejection fraction [LVEF]<40%), who are in sinus rhythm or who will be cardioverted). Dronedarone is also found to be useful in prevention of stroke or transient ischemic attack, prevention of permanent atrial fibrillation, prevention of cardioversion, regulating potassium levels in blood, for prevention of cardiac arrhythmia and increased creatinine level, reducing death rate after infarction and reducing death rate after infarction. The primary advantage of dronedarone is its comparatively lower side-effect profile vis-à-vis amiodarone. Due to high presystemic first pass metabolism the absolute oral bioavailability is only 4% (fasting) which increases to approx. 15% when administered with a high fat meal.

U.S. Pat. No. 5,223,510 assigned to Sanofi discloses dronedarone specifically.

U.S. Pat. No. 7,323,493 assigned to Sanofi Aventis relates to a solid pharmaceutical composition for oral administration characterized in that it comprises a benzofuran derivative with antiarrhythmic activity, or one of the pharmaceutically acceptable salts thereof, as an active principle, and a pharmaceutically acceptable nonionic hydrophilic surfactant optionally in combination with one or more pharmaceutical excipients.

US 2007/0243257 filed by Sanofi Aventis relates to a solid pharmaceutical composition comprising a solid dispersion containing at least one active principle and a pharmaceutically acceptable polymer matrix, characterized in that said pharmaceutically acceptable polymer matrix comprises a blend of (i) polydextrose, in the form of a continuous polydextrose phase, in order to promote the disintegration of the composition in an aqueous medium, and (ii) at least one polymer other than polydextrose, in the form of a continuous phase of this polymer, whereby the polydextrose is in a concentration of at least 20 wt % and the at least one polymer other than polydextrose is in a concentration of at least 20 wt % in relation to the total weight of said pharmaceutically acceptable polymer matrix.

US 2008/0139645 filed by Sanofi Aventis relates to a solid pharmaceutical composition for oral administration characterized in that it comprises a benzofuran derivative with antiarrhythmic activity, or one of the pharmaceutically acceptable salts thereof, as an active principle, and a pharmaceutically acceptable nonionic hydrophilic surfactant optionally in combination with one or more pharmaceutical excipients.

Although number of approaches have been disclosed in the prior art for preparing a formulation comprising dronedarone none describe a controlled relase formulation of dronedarone. There exists a need for controlled release formulation of Dronedarone which controls the release of Dronedarone in such a manner that therapeutically effective concentration is maintained in the blood for an extended period of time keeping the drug concentration in the blood substantially constant. Dronedarone has low solubility in aqueous media and/or at low pH, also at higher pH condition it precipitates out. As a result it has low in-vivo bioavailability. The use of controlled release formulations of Dronedarone would improve the bioavailability and the patient compliance with reduction in number of dosages to be taken per day.

### SUMMARY OF THE INVENTION

One embodiment discloses a controlled release formulation of dronedarone comprising: dronedarone, a controlled release polymer and pharmaceutically acceptable excipients.

Another embodiment discloses a controlled release formulation comprising:

(i) Dronedarone, controlled release polymer and pharmaceutically acceptable excipients,
(ii) Controlled release coating.

Another embodiment discloses a controlled release formulation of dronedarone comprising dronedarone along with pharmaceutically acceptable excipients and controlled release coating.

Another embodiment discloses a controlled release formulation comprising:

(i) Dronedarone and pharmaceutically acceptable excipients,
(ii) One or more coating optionally comprising dronedarone.

Another embodiment discloses a controlled release formulation of dronedarone wherein the controlled release polymer used may be bioadhesive.

### BRIEF DESCRIPTION OF THE DRAWINGS

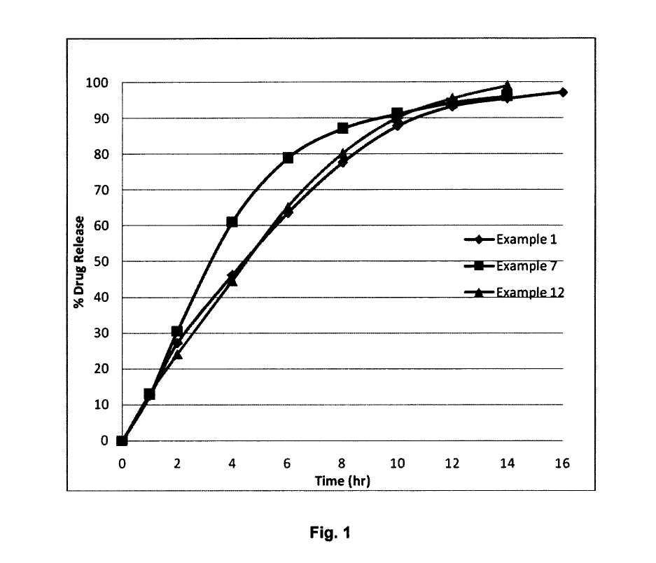

Drug release for examples 1, 7 and 12 are reproduced in the attached FIG. 1

### DETAILED DESCRIPTION OF THE INVENTION

A controlled release formulation of dronedarone or pharmaceutically acceptable salts, esters, metabolites, prodrugs or enantiomers thereof and pharmaceutically acceptable excipients.

The term “formulation” as used herein refers to the drug with pharmaceutically acceptable excipients. This includes orally administrable formulations as well as formulations administrable by other means.

“Controlled release formulation” as used herein are those whose drug release characteristics of time course and/or location are chosen to accomplish therapeutic or convenience objectives not offered by conventional, immediate release dosage forms.

“Controlled release formulation” or dosage forms which exhibit a “controlled release” used herein is defined to mean formulations that release the drug at a controlled rate and provide plasma concentrations of the drug that remain controlled with time within the therapeutic range of the drug over a 24-hour period. “Controlled release” is defined to mean release of the drug gradually or in a controlled manner per unit time. For example, the controlled rate can be a constant rate providing plasma concentrations of the drug that remain invariant with time within the therapeutic range of drug over at least a 24-hour period.

The term controlled release formulation may be used interchangeably with prolonged release formulation, programmed release formulation, timed release formulation, modified release formulation, site specific release formulation, sustained release formulation, extended release formulation, slow release formulation, pulsatile release formulation, delayed release formulation. The controlled release formulations can be orally disintegrating extended release formulation, osmotic dosage form, bioadhesive formulation, gastroretentive formulation and other such dosage forms.

As used herein the term dronedarone includes all forms of dronedarone or pharmaceutically acceptable salts, esters, solvates, hydrates, metabolites, prodrugs or isomers thereof. The most preferred form is dronedarone hydrochloride.

The controlled release formulation may be in the form of tablets (single layered tablets, multilayered tablets, mini tablets, bioadhesive tablets, floating formulation, caplets, matrix tablets, tablet within a tablet, mucoadhesive tablets, modified release tablets, pulsatile release tablets, gastroretentive tablets and timed release tablets), pellets, beads, granules, spheroids, particles, compact, powders, capsules, microcapsules, tablets in capsules, microspheres, matrix formulations, and microencapsulation.

The term “pharmaceutically-acceptable excipients” as used herein includes any physiologically inert, pharmacologically inactive material known to one skilled in the art, which is compatible with the physical and chemical characteristics of Dronedarone.

One embodiment discloses a controlled release formulation comprising: dronedarone, controlled release polymer and pharmaceutically acceptable excipients.

The controlled release formulation of dronedarone may contain one or more than one controlled release polymer.

Other embodiment discloses a controlled release formulation comprising dronedarone having particle size (D₉₀) less than 100 microns. Preferably the particle size (D₉₀) of dronedarone is less than 60 microns and more preferably particle size (D₉₀) less than 30 micron.

The controlled release polymer may be selected from water soluble polymer, water insoluble polymer, waxy material or combination thereof.

The water soluble polymer may be selected from alkyl celluloses such as methyl cellulose; hydroxyalkyl celluloses, for example, hydroxymethyl cellulose, hydroxyethyl cellulose, hydroxypropyl cellulose, and hydroxybutyl cellulose; hydroxyalkyl alkyl celluloses such as hydroxyethyl methyl cellulose and hydroxypropyl methyl cellulose; sodium or calcium carboxymethyl cellulose, methyl ethyl cellulose, ethylhydroxy ethylcellulose, carboxyalkyl cellulose esters; carbomers; glycerol fatty acid esters, sorbitan esters, lecithins, other natural, semi-synthetic, or synthetic di-, oligo-, and polysaccharides such as galactomannans, tragacanth, agar, guar gum, gum arabic, pectin, acacia, karaya, locust bean gum, xanthan gum, pullulan, collagen, casein, carrageenan, aligns, polycarbophil, ammonia alginate, sodium, calcium, potassium alginates, propylene glycol alginate, scleroglucan and polyfructans, maltodextrin; methacrylate copolymers; polyvinyl alcohol; polyvinylpyrrolidone, copolymers of polyvinylpyrrolidone with vinyl acetate; combinations of polyvinyl alcohol and polyvinylpyrrolidone; and polyalkylene oxides such as polyethylene oxide and polypropylene oxide and copolymers of ethylene oxide and propylene oxide, carboxyvinyl polymers, sodium alginate, sodium hyluronate, sodium carmellose, calcium carmellose, sodium carboxymethyl starch, gelatin, starch, crosslinked starch, microcrystalline cellulose, ceratonia, chitin, poly(hydroxyalkyl methacrylate), polyvinyl alcohol having a low acetate residual, a swellable mixture of agar and carboxymethyl cellulose, crosslinked polyvinyl alcohol and poly N-vinyl-2-pyrrolidone and mixtures and blends thereof.

Water insoluble polymer may be selected from cellulose acylate; cellulose ethyl ether; cellulose diacylate; cellulose triacylate; cellulose acetate; cellulose diacetate; cellulose triacetate; mono-, di- and tricellulose alkan, mono-, di- and tricellulose aroyl; ethyl cellulose; cellulose acetate; cellulose acetate butyrate; cellulose acetate phthalate; cellulose acetate trimellitate; glyceryl monooleate; glyceryl monostearate; glyceryl palmitostearate; polyvinyl acetate phthalate; hydroxypropylmethylcellulose phthalate; hydroxypropylmethylcellulose acetate succinate; poly(alkyl methacrylate); poly(vinyl acetate); poly vinyl alcohols; polyacrylamide derivatives ammonio methacrylate copolymers, poly acrylic acid and poly acrylate and methacrylate copolymers, aminoacryl-methacrylate copolymer, polyvinyl acetaldiethylamino acetate, copolymers of maleic anhydride and styrene, ethylene, propylene or isobutylene, polyacrylamides, polyox(polyethylene oxides), diesters of polyglucan, cellulose butyrate, cellulose propionate, shellac, chitosan, oleyl alcohol, zein, hydrogenated castor oil and the like.

Waxy material may be selected from carnauba wax; beeswax; chinese wax; spermaceti; lanolin; bayberry wax; white wax; yellow wax; candelilla wax; microcrystalline wax; castor wax; esparto wax; Japan wax; jojoba oil; cotton seed oil, corn oil, hydrogenated cotton seed oil, ouricury wax; rice bran wax; ceresin waxes; montan wax; ozokerite; peat waxes; paraffin wax; polyethylene waxes; and polyglycerol fatty acid esters.

One embodiment discloses a controlled release formulation of dronedarone comprising: dronedarone, a controlled release polymer and pharmaceutically acceptable excipients.

Another embodiment discloses a controlled release formulation of dronedarone wherein the controlled release polymer used may be bioadhesive.

The bioadhesive polymers may be selected from proteins (e.g., hydrophilic proteins) such as carbomers, pectin, zein, modified zein, casein, gelatin, gluten, serum albumin and collagen; chitosan; oligosaccharides; polysaccharides such as cellulose, dextrans, tamarind seed polysaccharide, gellan, carrageenan, xanthan gum, gum arabic, hyaluronic acid, polyhyaluronic acid, alginic acid and sodium alginate; glyceryl monooleate; polyamides; polycarbonates; polyalkylenes; polyalkylene glycols; polyalkylene oxides; polyalkylene terephthalates; polyvinyl alcohols; polyvinyl ethers; polyvinyl esters; polyvinyl halides; polyvinylpyrrolidone; polyglycolides; polysiloxanes; polyurethanes; polystyrene; polymers of acrylic and methacrylic esters; polylactides; poly(butyric acid); poly(valeric acid); poly(lactide-co-glycolide); polyanhydrides; polyorthoesters; poly(fumaric acid); poly(maleic acid); poly(methyl vinyl ether/maleic anhydride); polycarbophil and blends or copolymers or mixtures thereof.

The controlled release formulation may further contain one or more pharmaceutically acceptable excipients such as binders; diluents; lubricants; disintegrating agents; glidants; stabilizers; osmotic agents; dissolution enhancing agents; and surface active agents.

Examples of binders include, potato starch; pregelatinized starch; modified starch; gelatin; wheat starch; corn starch; celluloses such as methyl cellulose, hydroxypropyl cellulose, hydroxyethyl cellulose, hydroxypropylmethyl cellulose, ethyl cellulose and sodium carboxy methyl cellulose; hydroxypropyl Starch, polymethacrylates; carbomers; natural gums such as acacia, alginic acid and guar gum; lactose (anhydrous, monohydrate, spraydried); liquid glucose; dextrin; sodium alginate; kaolin; povidone; syrup; polyethylene oxide; polyvinyl pyrrolidone; poly vinyl alcohol; poly-N-vinyl amide; polyethylene glycol; sucrose; polydextrose; gelatin; poly propylene glycol; tragacanth; ceratonia; glyceryl behenate; hydrogenated vegetable oil; zein; castor oil; paraffin; higher aliphatic alcohols; higher aliphatic acids; long chain fatty acids; fatty acid esters; agar; chitosan; maltodextrin; magnesium aluminum silicate; inulin and wax-like materials such as fatty alcohols, fatty acid esters, fatty acid glycerides, hydrogenated fats, hydrocarbons, stearic acid; copovidone; dextrates, sunflower oil and stearyl alcohol.

Examples of diluents include microcrystalline cellulose; lactose, cellulose powdered, cellulose silicified, cellulose acetate, methyl cellulose, microcrystalline lactose; dibasic or tribasic calcium phosphate; saccharides; confectioner's sugar; compressible sugar; confectioner's sugar; sugar spheres; dextrates; dextrin; dextrose; fructose; maltose; sodium chloride; lactitol; maltodextrin; mannitol; sucrose; fructose; glyceryl palmitostearate; semithicone; Magnesium aluminum silicate; starch; pregelatinized starch; maltitol; xylitol; erythritol; isomalt; sorbitol; sulfobutylether b-cyclodextrin, polymethacrylates; talc; trehalose; ammonium alginate; calcium carbonate; ethyl cellulose; magnesium carbonate; magnesium oxide and calcium sulphate.

The disintegrating agents include povidone, low-substituted hydroxypropyl cellulose; cross-linked polyvinyl pyrrolidone; cross-linked sodium carboxymethylcellulose; hydroxypropyl starch; sodium starch glycolate; sodium starch glucolate; sodium carboxymethylcellulose; carboxymethyl cellulose calcium; sodium carboxymethyl starch; ion-exchange resins such as polacrillin potassium; microcrystalline cellulose; starches and pregelatinized starch; formalin-casein; clays such as bentonite or veegum; guar gum; celluloses or cellulose derivatives; sodium alginate; calcium alginate; alginic acid; chitosan; magnesium aluminum silicate; colloidal silicon dioxide.

The lubricants may be selected from Mg, Al, Ca or Zn stearate; polyethylene glycol; polyvinyl alcohol; glyceryl behenate; glyceryl monostearate; Glyceryl palmitostearate; potassium benzoate; sodium benzoate; mineral oil; sodium stearyl fumarate; palmitic acid, myristic acid; stearic acid; hydrogenated vegetable oil; hydrogenated castor oil; talc; hydrogenated soybean oil; stearyl alcohol; leucine; sodium lauryl sulfate; ethylene oxide polymers; poloxamer; octyldodecanol; Sodium stearyl fumarate and colloidal silica.

The stabilizers may be selected from naturally occurring as well as synthetic phospholipids, their hydrogenated derivatives and mixtures thereof; organic acids like acetic acid, tartaric acid, citric acid, fumaric acid, lactic acid, and mixtures thereof sphingolipids and glycosphingolipids; physiological bile salts such as sodium cholate, sodium dehydrocholate, sodium deoxycholate, sodium glycocholate and sodium taurocholate; saturated and unsaturated fatty acids or fatty alcohols; ethoxylated fatty acids or fatty alcohols and their esters and ethers; alkylaryl-polyether alcohols such as tyloxapol; esters and ethers of sugars or sugar alcohols with fatty acids or fatty alcohols; acetylated or ethoxylated mono- and diglycerides; synthetic biodegradable polymers like block co-polymers of polyoxyethylene and polyoxypropyleneoxide; ethoxylated sorbitanesters or sorbitanethers; amino acids, polypeptides and proteins such as gelatine and albumin; or combination thereof.

The glidants may be selected from magnesium trisilicate; powdered cellulose; starch; talc; tribasic calcium phosphate; calcium silicate; magnesium silicate; magnesium trisilicate; colloidal silicon dioxide; and silicon hydrogels.

Dissolution enhancing agents may be selected from, but are not limited to, organic acids, inorganic acids or combination thereof. The organic acids include, but not limited to citric acid, fumaric acid, malic acid, maleic acid, tartaric acid, succinic acid, oxalic acid, aspartic acid, mandelic acid, glutaric acid, and glutamic acid. The inorganic acids include but not limited to hydrochloric acid, phosphoric acid, nitric acid, and sulfuric acid.

The surface active agents used may be hydrophilic, hydrophobic or combination thereof.

Hydrophilic surfactants may be either ionic or non-ionic.

Suitable hydrophilc ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; ammonium lauryl sulfate, sodium lauryl sulfate, sodium myreth sulfate, dioctyl sodium sulfosuccinate, perfluorooctanesulfonate, perfluorobutanesulfonate, alkyl benzene sulfonates, alkyl aryl ether phosphate, alkyl ether phosphate, alkyl carboxylates like, fatty acid salts, sodium stearate, sodium lauroyl sarcosinate, octenidine dihydrochloride, cetyl trimethylammonium bromide (CTAB) or hexadecyl trimethyl ammonium bromide, cetyl trimethylammonium chloride (CTAC), cetylpyridinium chloride (CPC), polyethoxylated tallow amine (POEA), benzalkonium chloride (BAC), benzethonium chloride (BZT), 5-Bromo-5-nitro-1,3-dioxane, dimethyldioctadecylammonium chloride, dioctadecyldimethylammonium bromide (DODAB), cocamidopropyl betaine, cocamidopropyl hydroxysultaine and mixtures thereof.

Suitable hydrophilic non-ionic surfactants include alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof.

Suitable lipophilic surfactants include, but are not limited to fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

The osmotic agents may be selected from sodium chloride; potassium chloride; magnesium sulfate; magnesium chloride; sodium sulfate; lithium sulfate; urea; inositol; sucrose; lactose (anhydrous, monohydrate, spraydried); glucose; sorbitol; fructose; mannitol; dextrose; magnesium succinate; and potassium acid phosphate, sulfobutylether b-cyclodextrin. The osmotic agents may also be added in the controlled release coating.

Another embodiment discloses a controlled release formulation comprising:

(i) Dronedarone, and pharmaceutically acceptable excipients,
(ii) Controlled release coating.

The controlled release coating may be functional coating; moisture barrier coatings; enteric polymeric coatings; sustained release coating; and the like.

Another embodiment discloses a controlled release formulation comprising:

(i) Dronedarone, controlled release polymer and pharmaceutically acceptable excipients,
(ii) Controlled release coating.

Another embodiment discloses a controlled release formulation comprising:

(i) Dronedarone and pharmaceutically acceptable excipients,
(ii) One or more coating optionally comprising dronedarone.

The coating may be controlled release or enteric which would allow dronedarone to be released in lower GIT or an immediate release, optionally comprising dronedarone.

The controlled release coating comprises of controlled release polymer and other pharmaceutically acceptable excipients. The controlled release polymers includes hydrophilic polymers, hydrophobic polymers or waxes as disclosed above.

The pharmaceutically acceptable excipients that may be added to controlled release coating may include pore forming agents, lubricants, plasticizers and colorants.

The porosity of the controlled release coating may be modified by using pore forming agents. The pore forming agents may be polymeric or non polymeric in nature. Any water soluble material present in the coating which dissolves and forms pores in the coating layer may act as pore forming agents. Pore forming agents may be selected form of potassium salts such as potassium chloride, sodium salts as sodium chloride, calcium salts, magnesium salts, amino acids, weak acids, carbohydrates such as sucrose; mannitol; sorbitol, lactose (anhydrous, monohydrate, spraydried), polymers with amino and/or acid functions or polyvinyl pyrrolidine. Examples are aspargine, glutamine, leucin, neroleucine, meglumine, isoleucine, magnesium citrate, magnesium phosphate, magnesium carbonate, magnesium hydroxide, magnesium oxide.

Plasticizers include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

The lubricants used in coating include Mg, Al or Ca or Zn stearate; polyethylene glycol; polyvinyl alcohol; glyceryl behenate; glyceryl monostearate; Glyceryl palmitostearate; potassium benzoate; sodium benzoate; mineral oil; sodium stearyl fumarate; palmitic acid, myristic acid; stearic acid; hydrogenated vegetable oil; hydrogenated castor oil; talc; hydrogenated soybean oil; stearyl alcohol; leucine; sodium lauryl sulfate; ethylene oxide polymers; poloxamer; Octyldodecanol; Sodium stearyl fumarate and colloidal silica.

The controlled release formulation may be manufactured by various methods known in the art such as by dry granulation, slugging, roller compaction, wet granulation (using aqueous/nonaqueous solvents), melt granulation, solid dispersion, direct compression, double compression, extrusion spheronization, layering, High shear mixture granulation, Fluid bed granulation, spray drying, steam granulation, moisture activated dry granulation, moist granulation, thermal adhesion granulation, foam granulation and the like. Compaction of the blend into coprimate may be carried out using a slugging technique or roller compaction. The milling of the granules may be carried out according to conventional milling methods.

The solvent which may be used for manufacturing the formulation may be aqueous, non aqueous or combination thereof.

Melt granulation technique involves melting of the carrier at a higher temperature. The carrier for melt granulation may be selected from different grades of polyethylene glycols, cellulose ethers and acrylates, various molecular weights of polyethylene oxides, poly methacrylate derivatives, poloxamers, thermoplastic aliphatic poly(esters) such as poly(lactide) (PLA), poly(glycolide) (PGA) and copolymer of lactide and glycolide, poly(lactide-co-glycolide) (PLGA). Starch and starch derivatives, sugars and sugar alcohols and waxes.

Another embodiment discloses a process of manufacturing controlled release formulation of dronedarone.

Yet another embodiment discloses a bilayer tablet formulation wherein one layer is immediate release layer and the other layer is controlled release layer.

Further embodiment discloses a bilayer tablet formulation wherein both the layers are controlled release layers.

The coating operation may be conducted in standard equipment such as a fluid bed coater, a wurster coater or a rotary bed coater. The controlled release coating may be aqueous, nonaqueous or combination of the two.

The invention is not to be limited in scope by the specific embodiments described herein. Indeed, various modifications of the invention in addition to those described herein will become apparent to those skilled in the art from the foregoing description. Such modifications are intended to fall within the scope of the appended claims.

### Example 1

| Ingredients | % w/w* |
|---|---|
| Dronedarone HCl eq to Dronedarone | 70.0 |
| Polyvinyl pyrrolidone | 4.0 |
| Hydroxy propyl methyl cellulose | 15.0 |
| Lactose monohydrate | 10.0 |
| Magnesium Stearate | 1.0 |
| Purified water | q.s. |
| Film coat | For 2-5% wt. gain |

*As percentage of the core tablet weight

Procedure: Dronedarone hydrochloride, microcrystalline cellulose and lactose monohydrate were sifted and granulated using aqueous solution of polyvinyl pyrrolidone. The granules were dried, sifted and mixed with hydroxy propyl methyl cellulose. The granules were then lubricated and compressed. The compressed tablets were coated using film coating dispersion.

### Example 2

| Ingredients | % w/w* |
|---|---|
| Dronedarone HCl eq to Dronedarone | 70.0 |
| Polyvinyl pyrrolidone | 4.0 |
| Fumaric acid | 2.0 |
| Hydroxy propyl methyl cellulose | 15.0 |
| Lactose monohydrate | 8.0 |
| Magnesium Stearate | 1.0 |
| Purified water | q.s. |
| Film coat | For 2-5% wt. gain |

*As percentage of the core tablet weight

Procedure: Dronedarone hydrochloride, microcrystalline cellulose, fumaric acid and lactose monohydrate were sifted and granulated using aqueous solution of polyvinyl pyrrolidone. The granules were dried, sifted and mixed with hydroxy propyl methyl cellulose. The granules were then lubricated and compressed. The compressed tablets were coated using film coating dispersion.

### Example 3

| Ingredients | % w/w* |
|---|---|
| Dronedarone HCl eq to Dronedarone | 70.0 |
| Polyvinyl pyrrolidone | 4.0 |
| Fumaric acid | 2.0 |
| Docusate sodium | 1.5 |
| Hydroxy propyl methyl cellulose | 15.0 |
| Lactose monohydrate | 6.5 |
| Magnesium Stearate | 1.0 |
| Purified water | q.s. |
| Film coat | For 2-5% wt. gain |

*As percentage of the core tablet weight

Procedure: Dronedarone hydrochloride, microcrystalline cellulose, fumaric acid, docusate sodium and lactose monohydrate were sifted and granulated using aqueous solution of polyvinyl pyrrolidone. The granules were dried, sifted and mixed with hydroxy propyl methyl cellulose. The granules were then lubricated and compressed. The compressed tablets were coated using film coating dispersion.

### Example 4

| | |
|---|---|
| Ingredients | % w/w |

First Layer

| | |
|---|---|
| Dronedarone HCl eq to Dronedarone | 20 |
| Sterotex | 8.5 |
| Ethyl cellulose | 4.5 |
| Polyethylene glycol | 2.0 |
| Stearic acid | 1.0 |

Second Layer

| | |
|---|---|
| Dronedarone HCl eq to Dronedarone | 25.0 |
| Hydroxy propyl methyl cellulose | 10.0 |
| Xanthan gum | 8.0 |
| Lactose | 7.0 |
| Povidone | 1.5 |

Extra-granular portion

| | |
|---|---|
| Lactose monohydrate | 11.0 |
| Colloidal silicon dioxide | 1.0 |
| Magnesium stearate | 0.5 |

### Procedure:

I. Sterotex and polyethylene glycol were melted and to this molten mass, dronedarone hydrochloride and stearic acid were added with stirring, until a uniform mass is formed. The molten uniform mass was cooled to room temperature and milled. The granules thus obtained were sifted using suitable sieve.
II. Dronedarone hydrochloride was mixed with lactose, and granulated with povidone solution. Mix the dried granules with hydroxy propyl methyl cellulose and xanthan gum.
III. The granules of step I and II were mixed with lactose monohydrate and silicon dioxide separately. The granules of Step I and II were then lubricated separately. The two lubricated blends were compressed into bilayered tablets.

### Example 5

| Ingredients | % w/w |
|---|---|
| Dronedarone HCl eq to Dronedarone | 65.0 |
| Polyvinyl pyrrolidone | 4.0 |
| Sodium lauryl sulfate | 1.5 |
| Lactose monohydrate | 3.5 |
| Microcrystalline cellulose | 5.0 |
| Magnesium stearate | 1.0 |
| Purified water | q.s. |
| Ethyl cellulose | 10.0 |
| Polyethylene glycol | 10.0 |
| Isopropyl alcohol/dichloromethane/acetone mixture | q.s. |

Procedure: Dronedarone hydrochloride, microcrystalline cellulose, sodium lauryl sulfate and lactose monohydrate were sifted and granulated using solution of polyvinyl pyrrolidone. The granules were dried and sifted. The granules were then lubricated and compressed.

The compressed tablets were coated using solution of ethyl cellulose and polyethylene glycol using its non-aqueous solution

### Example 6

| Ingredients | % w/w* |
|---|---|
| Dronedarone HCl eq to Dronedarone | 60.0 |
| Polyvinyl pyrrolidone | 4.0 |
| Hydroxy propyl methyl cellulose | 5.0 |
| Sterotex | 5.0 |
| Lactose monohydrate | 5.0 |
| Microcrystalline cellulose | 5.0 |
| Magnesium Stearate | 1.0 |
| Purified water | q.s. |
| Ethyl cellulose | 10.0 |
| Polyethylene glycol | 0-5.0 |
| Isopropyl alcohol/dichloromethane/acetone mixture | q.s. |

*As percentage of the core tablet weight

Procedure: Dronedarone hydrochloride, microcrystalline cellulose and lactose monohydrate were sifted and granulated using aqueous solution of polyvinyl pyrrolidone. The granules were dried and sifted. The dried granules were mixed with hydroxy propyl methyl cellulose and sterotex. The granules were then lubricated and compressed. The compressed tablets were coated using solution of ethyl cellulose and polyethylene glycol using their non-aqueous solution.

### Example 7

| | |
|---|---|
| Ingredients | % w/w |

Active Layer

| | |
|---|---|
| Dronedarone HCl eq to Dronedarone | 65.0 |
| Polyvinyl pyrrolidone | 2.5 |
| Hydroxy propyl methyl cellulose | 5.0 |
| Lactose Monohydrate | 5.5 |
| Eudragit | 5.5 |
| Micro crystalline cellulose | 3.0 |
| Magnesium stearate | 0.5 |
| Purified water | q.s. |

Bio-adhesive Layer

| | |
|---|---|
| Sterotex | 2.0 |
| Eudragit | 2.0 |
| Polyethylene oxide | 3.0 |
| Hydroxy propyl methyl cellulose | 5.0 |
| Silicon dioxide | 0.5 |
| Magnesium stearate | 0.5 |

### Procedure:

I. Dronedarone hydrochloride was granulated along with sterotex, hydroxy propyl methyl cellulose, Eudragit, lactose and microcrystalline cellulose using polyvinyl pyrrolidone. The granules were dried, sifted and lubricated.
II. Sterotex, Eudragit, polyethylene oxide and hydroxy propyl methyl cellulose were sifted and mixed.
III. The blend of step I and II were then lubricated separately and compressed into bilayer tablets.

### Example 8

| Ingredients | % w/w |
|---|---|
| Dronedarone HCl eq to Dronedarone | 54.0 |
| Polyvinyl pyrrolidone | 3.0 |
| Mannitol | 15.0 |
| Sodium lauryl sufate | 2.0 |
| Lactose monohydrate | 5.0 |
| Microcrystalline cellulose | 4.5 |
| Magnesium stearate | 0.5 |
| Purified water | q.s. |
| Cellulose acetate | 10.0 |
| Polyethylene glycol | 5.0 |
| Triacetine | 1.0 |
| Acetone | q.s. |

### Procedure:

Dronedarone hydrochloride, mannitol, sodium lauryl sulfate and lactose monohydrate were sifted and granulated using aqueous solution of polyvinyl pyrrolidone. The granules were dried, sifted and mixed with microcrystalline cellulose. The blend was lubricated using magnesium stearate. The lubricated blend was compressed using round shaped punches of suitable size and coated with solution of cellulose acetate, triacetin and polyethylene glycol in acetone. Orifice was drilled on coated tablets using laser drilling technology.

### Example 9

| | |
|---|---|
| Ingredients | % w/w |

Active layer granules of core

| | |
|---|---|
| Dronedarone HCl eq to Dronedarone | 65.0 |
| Polyvinyl pyrrolidone | 3.0 |
| Mannitol | 6.5 |
| Sodium lauryl sulfate | 2.0 |
| Polyethylene oxide | 3.0 |
| Magnesium stearate | 0.5 |
| Isopropyl alcohol | q.s. |

Push-pull layer granules of core

| | |
|---|---|
| Poly ethylene oxide | 5.0 |
| Potassium chloride | 5.0 |
| Hypromellose | 2.0 |
| Hydroxy propyl cellulose | 1.0 |
| Magnesium stearate | 0.5 |

Coating (5-15% wt.gain)

| | |
|---|---|
| Cellulose acetate | 5.0 |
| Polyethylene glycol | 1.0 |
| Triacetine | 0.5 |
| Acetone | q.s. |

### Procedure:

I. Dronedarone HCl was mixed with mannitol, sodium lauryl sulfate and polyethylene oxide, and granulated with solution of povidone and hypromellose in isopropyl alcohol. The granules were dried, sifted, lubricated.
II. Polyethylene oxide potassium chloride, hypromellose and hydroxy propyl cellulose was mixed and lubricated with magnesium stearate.
III. The granules of step I and II were compressed separately as bilayered tablet using suitable size and shape punch.
IV. The compressed tablets were coated with solution of cellulose acetate, triacetine and polyethylene glycol in acetone. Orifice was drilled on coated tablets using laser drilling technology.

### Example 10

| Ingredients | % w/w* |
|---|---|
| Dronedarone HCl eq to Dronedarone | 30.0 |
| D-alpha-tocopherol polyethylene glycol 1000 | 2.0 |
| succinate | |
| Steroyl polyoxyl glycerides | 25.0 |
| Polyethylene glycol | 10.0 |
| Hydroxy propyl methyl cellulose | 10.0 |
| Microcrystalline cellulose | 15.0 |
| Stearic acid | 5.0 |
| Colloidal silicon dioxide | 2.0 |
| Magnesium stearate | 1.0 |
| Film coating | For 2-3% wt |
| | gain |

*As percentage of the core tablet weight

Procedure: All ingredients were sifted through suitable sieve. Sterotex and polyethylene glycol was melted in preheated steam jacketed vessel at 60-70° C. and to this melted mass Dronedarone HCl and stearic acid was added under stirring. The heating was stopped with stirring continued for 30-45 min until a uniform mass is formed. The molten uniform mass is cooled to room temperature and milled in co-mill using suitable sieve. The milled and sieved mass was first mixed with microcrystalline cellulose and subsequently with colloidal silicon dioxide. The final blend was lubricated with magnesium stearate. The final blend was compressed into tablet using suitable punch tooling and coated with film coating solution.

### Example 11

| Ingredients | % w/w* |
|---|---|
| Dronedarone HCl eq to Dronedarone | 55.0 |
| Tocophersolan | 15.0 |
| Medium chain triglycerides (MCT) | 5.0 |
| Polyethylene glycol | 5.0 |
| Hydroxy propyl methyl cellulose | 5.0 |
| Microcrystalline cellulose | 5.0 |
| Colloidal silicon dioxide | 9.0 |
| Magnesium stearate | 1.0 |
| Film coating | For 2-3% wt gain |

Procedure: All ingredients were sifted through suitable sieve. Tocophersolan was melted in preheated steam jacketed vessel at 60-70° C. along with medium chain triglyceride and to this melted mass Dronedarone HCl, colloidal silicon dioxide and polyethylene glycol were added under stirring. The heating was stopped with stirring continued for 30-45 min until a uniform mass is formed. The molten uniform mass was cooled to room temperature and milled in co-mill using suitable sieve. The milled and sieved mass was first mixed with microcrystalline cellulose, hydroxy propyl methyl cellulose and subsequently with remaining colloidal silicon dioxide and lubricated. The final blend was compressed into tablet using suitable punch tooling and coated with film coating solution.

### Example 12

| Ingredients | % w/w* |
|---|---|
| Dronedarone HCl eq to Dronedarone | 58.90 |
| Co-povidone | 20.55 |
| Poly Ethylene Oxide | 2.05 |
| Maize Starch | 0.87 |
| Crospovidone | 3.08 |
| Monobasic Potassium Phosphate | 1.37 |
| Hypromellose | 12.14 |
| Dichloromethane | — |
| Colloidal silicon dioxide | 0.68 |
| Magnesium Stearate | 0.34 |
| Film Coating | For 2-3% weight |
| | gain |

*As percentage of the core tablet weight

Procedure: All ingredients were sifted through suitable sieve and granulated using Dichloromethane. The granules were dried and sifted. The dried granules were then first mixed with Crospovidone and subsequently with Colloidal silicon dioxide. The final blend was lubricated with Magnesium stearate. The lubricated blend was compressed into tablets using suitable punch tooling and coated with film coating solution.

### Example 13

| Ingredients | % w/w* |
|---|---|
| Dronedarone HCl eq to Dronedarone | 34.4 |
| Co-povidone | 12.1 |
| Lactose monohydrate | 1.6 |
| Maize Starch | 1.6 |
| Crospovidone | 2.8 |
| Colloidal Silicon dioxide | 0.4 |
| Magnesium stearate | 0.4 |
| Controlled Release Coating-I | |
| Hypromellose | 2.4 |
| Triethyl Citrate | 0.3 |

Over Coat - I

| | |
|---|---|
| Dronedarone HCl eq to Dronedarone | 17.2 |
| Co-povidone | 2.0 |
| Hypromellose | 0.5 |
| Triethyl Citrate | 0.4 |

Controlled Release Coating - II

| | |
|---|---|
| Hypromellose | 3.2 |
| Triethyl Citrate | 0.6 |

Over Coat - II

| | |
|---|---|
| Dronedarone HCl eq to Dronedarone | 17.2 |
| Co-povidone | 2.0 |
| Hypromellose | 0.5 |
| Triethyl Citrate | 0.4 |
| Total | 100 |

Procedure: All ingredients were sifted through suitable sieve and granulated using Dichloromethane. The granules were dried and sifted through suitable sieve. The dried granules were blended with crospovidone and lactose monohydrate followed with colloidal silicon dioxide. The lubricated blend was compressed into tablets using suitable punch tooling. The compressed tablet was coated with hypromellose and Methyl citrate to obtain suitable weight gain. The coated tablets were further over coated with drug dispersion to achieve suitable weight gain. The process of coating and drug over coating was repeated to achieve the desired drug loading.

### Example 14

| | |
|---|---|
| Ingredients | % w/w* |

Step I

| | |
|---|---|
| Dronedarone HCl eq to Dronedarone | 75.0 |
| Microcrystalline Cellulose | 7.5 |
| Lactose monohydrate | 7.5 |
| Hypromellose | 10.0 |
| Purified water | q.s |

Step II

| | |
|---|---|
| Dronedarone spheres of Step A | 70.0 |
| Hypromellose | 20.0 |
| Triethyl Citrate | 10.0 |
| Iso propyl Alcohol | q.s |
| Dichloromethane | q.s |

Step III

| | |
|---|---|
| Dronedarone ER pellets of Step B | 75.0 |
| Poly(meth)acrylates | 15.0 |
| Triethyl Citrate | 10.0 |
| Iso propyl Alcohol | q.s |
| Dichloromethane | q.s |

Step IV

| | |
|---|---|
| Dronedarone ER pellets of Step A | 65.0 |
| Poly(meth)acrylates | 25.0 |
| Triethyl Citrate | 10.0 |
| Iso propyl Alcohol | q.s. |
| Dichloromethane | q.s. |

Step V

| | |
|---|---|
| Active spheres of I | 18.0 |
| Active spheres of II | 18.0 |
| Active spheres of III | 23.0 |
| Active spheres of IV | 16.0 |
| Microcrystalline cellulose | 15.0 |
| Hydrogenated vegetable oil | 10.0 |
| Colloidal silicon dioxide | 3.0 |
| Magnesium stearate | 2.0 |

Procedure: All ingredients were sifted through suitable sieve and granulated with sufficient quantity of purified water. The wet mass was passed through extruder, spheronized and dried. Part of the prepared spheres was coated with coating polymer solution/dispersion to achieve desired weight gain. A separate part of the prepared spheres were coated with poly(meth)acrylates to achieve desired weight gain. All the actives spheres were combined and blended with microcrystalline cellulose, hydrogenated vegetable oil and colloidal silicon dioxide compressed into tablets using suitable punch tooling or filled in hard gelatin capsule.

### Dissolution Profile:

The dissolution of the controlled release formulation of example 1, 7 and 12 was carried out at 4.5 pH phosphate buffer, 1000 ml, USP I (Basket) at 100 rpm.

TABLE 1
Drug release Profile:

| Time (hr) | Example 1 | Example 7 | Example 12 |
|---|---|---|---|
| 0 | 0 | 0 | 0 |
| 1 | 12.6 | 12.8 | 13.1 |
| 2 | 27.2 | 30.5 | 24.1 |
| 4 | 46.2 | 61.1 | 44.6 |
| 6 | 63.5 | 78.8 | 65.2 |
| 8 | 77.7 | 87.1 | 80.2 |
| 10 | 87.8 | 91.2 | 90.1 |
| 12 | 93.2 | 94.2 | 95.5 |
| 14 | 95.5 | 96.1 | 99 |
| 16 | 97.2 | — | — |

1. A controlled release formulation of dronedarone comprising dronedarone, controlled release polymer and pharmaceutically acceptable excipients.

2. The controlled release formulation of claim 1 wherein the controlled release polymer may be selected from water soluble polymer, water insoluble polymer, waxy material or combination thereof.

3. The controlled release formulation of claim 2 comprising water soluble polymer may be selected from alkyl celluloses such as methyl cellulose; hydroxyalkyl celluloses, for example, hydroxymethyl cellulose, hydroxyethyl cellulose, hydroxypropyl cellulose, and hydroxybutyl cellulose; hydroxyalkyl alkyl celluloses such as hydroxyethyl methyl cellulose and hydroxypropyl methyl cellulose; sodium or calcium carboxymethyl cellulose, methyl ethyl cellulose, ethylhydroxy ethylcellulose, carboxyalkyl cellulose esters; carbomers; glycerol fatty acid esters, sorbitan esters, lecithins, other natural, semi-synthetic, or synthetic di-, oligo-, and polysaccharides such as galactomannans, tragacanth, agar, guar gum, gum arabic, pectin, acacia, karaya, locust bean gum, xanthan gum, pullulan, collagen, casein, carrageenan, aligns, polycarbophil, ammonia alginate, sodium, calcium, potassium alginates, propylene glycol alginate, scleroglucan and polyfructans, maltodextrin; methacrylate copolymers; polyvinyl alcohol; polyvinylpyrrolidone, copolymers of polyvinylpyrrolidone with vinyl acetate; combinations of polyvinyl alcohol and polyvinylpyrrolidone; and polyalkylene oxides such as polyethylene oxide and polypropylene oxide and copolymers of ethylene oxide and propylene oxide, carboxyvinyl polymers, sodium alginate, sodium hyluronate, sodium carmellose, calcium carmellose, sodium carboxymethyl starch, gelatin, starch, crosslinked starch, microcrystalline cellulose, ceratonia, chitin, poly(hydroxyalkyl methacrylate), polyvinyl alcohol having a low acetate residual, a swellable mixture of agar and carboxymethyl cellulose, crosslinked polyvinyl alcohol and poly N-vinyl-2-pyrrolidone and mixtures and blends thereof and pharmaceutically acceptable excipients.

4. The controlled release formulation of claim 2 comprising water insoluble polymer selected from cellulose acylate; cellulose ethyl ether; cellulose diacylate; cellulose triacylate; cellulose acetate; cellulose diacetate; cellulose triacetate; mono-, di- and tricellulose alkan, mono-, di- and tricellulose aroyl; ethyl cellulose; cellulose acetate; cellulose acetate butyrate; cellulose acetate phthalate; cellulose acetate trimellitate; glyceryl monooleate; glyceryl monostearate; glyceryl palmitostearate; polyvinyl acetate phthalate; hydroxypropylmethylcellulose phthalate; hydroxypropylmethylcellulose acetate succinate; poly(alkyl methacrylate); poly(vinyl acetate); poly vinyl alcohols; polyacrylamide derivatives ammonio methacrylate copolymers, poly acrylic acid and poly acrylate and methacrylate copolymers, aminoacryl-methacrylate copolymer, polyvinyl acetaldiethylamino acetate, copolymers of maleic anhydride and styrene, ethylene, propylene or isobutylene, polyacrylamides, polyox(polyethylene oxides), diesters of polyglucan, cellulose butyrate, cellulose propionate, shellac, chitosan, oleyl alcohol, zein, hydrogenated castor oil and the like and pharmaceutically acceptable excipients.

5. The controlled release formulation of claim 2 comprising waxy material selected from carnauba wax; beeswax; chinese wax; spermaceti; lanolin; bayberry wax; white wax; yellow wax; candelilla wax; microcrystalline wax; castor wax; esparto wax; Japan wax; jojoba oil; cotton seed oil, corn oil, hydrogenated cotton seed oil, ouricury wax; rice bran wax; ceresin waxes; montan wax; ozokerite; peat waxes; paraffin wax; polyethylene waxes; and polyglycerol fatty acid esters and pharmaceutically acceptable excipients.

6. A controlled release formulation of dronedarone comprising
(1) Dronedarone and pharmaceutically acceptable excipients and
(2) Controlled release coating.

7. The controlled release formulation of claim 6 wherein the controlled release coating comprises of controlled release polymer.

8. The controlled release formulation of claim 6 wherein the controlled release polymers includes hydrophilic polymers, hydrophobic polymers or waxes.

9. The controlled release formulation of claim 6 wherein the controlled release coating may be functional coating, moisture barrier coating, enteric polymeric coating, sustained release coating and the like.

10. A controlled release formulation of dronedarone comprising
(1) Dronedarone and pharmaceutically acceptable excipients and
(2) One or more coating optionally comprising dronedarone.

11. The controlled release formulation of claim 10 wherein one or more coating is selected from controlled release or enteric or immediate release.

12. The controlled release formulation of claim 10 wherein the coating comprises of water soluble polymer, water insoluble polymer, waxy material or combination thereof as exemplified in example 13 or 14.
